(19) United States
(12) Patent Application Publication
(10) Pub. No.: US 2009/0299430 A1
(43) Pub. Date: Dec. 3, 2009

(54) Method and device for stabilising disordered breathing

(75) Inventors: Ceri Davies, Essex (GB); Keith Willson, East Sussex (GB); Charlotte Manisty, London (GB); Darrel Francis, Harrow (GB)

Correspondence Address: DeMont & Breyer, LLC, 100 Commons Way, Ste. 250, Holmdel, NJ, 07733, US

(73) Assignee: IMPERIAL INNOVATIONS LIMITED, London SW7 2AZ (GB)

(21) Appl. No.: 12/297,974

(22) PCT Filed: Apr. 20, 2007
(86) PCT No.: PCT/GB07/01458

§ 371 Date: Jun. 4, 2009

(30) Foreign Application Priority Data
Apr. 21, 2006 (GB) 0607939.6

(51) Int. Cl. A61N 1/365; A61M 16/00

(52) U.S. Cl. 607/22; 128/204.23

(57) ABSTRACT

A device and method for improving the stability of a ventilation pattern of a patient (1) uses a sensor (4) for sensing a parameter which reflects a level of lung gas in a patient, such as oxygen or carbon dioxide. The output signal of the sensor is received by a processor (3) which assesses the level of lung gas of the patient and activates means (18,20) for increasing the lung gas level of the patient beyond what it would otherwise have been without treatment in response to a decreasing level or a predicted decreasing level of the lung gas. Thus the device can be used to retard a decrease in said lung gas level, thereby reducing oscillations in the respiration.

The present invention relates to a method and device for stabilising disordered breathing resulting from cardiorespiratory control disorders.

There are several known disorders of respiratory control comprising of cyclical fluctuations in ventilation rate and depth of breathing. These include obstructive sleep apnoea (OSA), central sleep apnoea (CSA), Cheyne-Stokes respiration and periodic breathing (PB) in heart failure patients and idiopathic central apnoea. These all result in changes of respiratory parameters with peaks and troughs causing periods of shallow and sometimes slow breathing, sometimes followed by abnormally deep and rapid breaths. The fluctuations can be significant enough to result in episodes of complete cessation of respiration, called apnoeas. Associated with the oscillations in ventilation are consequent changes in the levels of carbon dioxide and oxygen in the blood (due to alterations in the net delivery of gases to and from the lungs), and also fluctuations in cardiac variables including blood pressure, heart rate and cardiac output.

Periodic breathing (PB) is a cyclic modulation of respiratory flow with a period of the order of one minute, and is a phenomenon seen in patients with heart failure (a state of impaired contraction of the heart muscle resulting in the cardiac output being insufficient to match metabolic demand). Periodic breathing is a strong negative prognostic indicator in congestive heart failure (CHF), but it is only relatively recently that the incidence and prognostic importance of PB has been recognised.

Sleep apnoea is defined as the cessation of breathing during sleep and is broadly divided into 2 types, obstructive sleep apnoea (OSA) and central sleep apnoea (CSA), the initiating mechanisms of which are entirely distinct. Many patients however have a mixture of the two types, or alternate between the two types. Both types of apnoea result not only in fluctuations of cardiorespiratory physiological parameters (e.g. heart rate, blood pressure, blood oxygen and carbon dioxide levels) but also in arousal of the patients from sleep, daytime somnolence, depression and decreased cognitive function. The nocturnal arousals last only short periods, but can prevent the person from achieving deep sleep (rapid eye movement and stage 3-4 sleep), which is necessary for satisfactory rest.

OSA typically involves episodes of snoring culminating in absent airflow, considered to be caused by an anatomical abnormality of the pharynx. The result is repetitive pauses in breathing during sleep due to the collapse/obstruction of the upper airway, which in turn causes reductions in blood oxygen saturation.

CSA is generally defined as a cessation of almost all respiratory effort during sleep, but with airway patency maintained. This type of sleep apnoea encompasses Cheyne-Stokes breathing and is also common in patients with CHF.

Patients with either periodic breathing or sleep apnoea have increased cardiovascular morbidity secondary to their respiratory problems, including systemic hypertension, pulmonary hypertension, stroke and cardiac arrhythmias and congestive heart failure.

Apnoeic disorders (a term that encompasses all of the above conditions) have been treated using various methods and devices, including surgery (uvulopalatopharyngoplasty), medication and respiratory mechanisms involving occlusive face masks or nasal devices that maintain a positive pressure in the respiratory tract (“CPAP”). These treatments have a low success rate. For example, only about 40% to 60% of uvulopalatopharyngoplasty patients show an improvement, and the surgery eliminates the apnoeic disorder in only about 10% of patients. Due to the use of pressurised gas to maintain a positive pressure in the respiratory tract, patients using respiratory mechanisms find the devices uncomfortable to wear and noisy, causing disturbed sleep. Side effects include nightmares, dry nose, nosebleeds and headaches. Consequently, patients do not comply with instructions to wear the device all night, with about 20% of patients refusing to even try treatments, and compliance rates of only about 40% in those subjects who do.

Many patients with CHF have implantable cardiac devices such as pacemakers and implantable cardioverters and defibrillators. These have a variety of functions for this patient group including improving the overall pumping ability of the heart, preventing the heart from beating too slowly, and shocking the patient out of dangerous heart rhythms should these occur. Recent evidence has suggested that increasing a patient's heart rate by manipulating the programming of their cardiac pacemaker may alleviate central and obstructive sleep apnoeas. However, simply pacing the heart at higher rates has two limitations. Firstly, it is effective in reducing apnoeic disorders only in patients with low heart rates rather than those with normal or high heart rates. Secondly, there are concerns that inducing an elevated heart rate may be detrimental the health of the patient.

U.S. Pat. No. 6,574,507 and U.S. Pat. No. 6,641,542 disclose proposals to treat sleep apnoeas by electrostimulation fundamentally by elevating heart rate for a prolonged period of time. The cardiac devices comprise one or more sensors to detect changes in physiological parameters e.g. HR, intrathorac impedance, or arterial oxygen saturations, which allows the detection of an apnoea. Both documents disclose monitoring the occurrence of apnoeas over a period of time. When more than a predetermined number of apnoeas per hour occur, treatment is initiated. During treatment, an electrostimulation is applied to accelerate the heart rate. U.S. Pat. No. 6,574,507 teaches raising the heart rate by at least 10 beats per minute above the patient's natural heart rate for 60 seconds. Afterwards, the heart rate is returned to its natural level. U.S. Pat. No. 6,641,542 teaches raising the heart rate by 15 beats per minute above the patient's mean nocturnal rate. That disclosure also teaches raising the patient's heart rate by between 5 and 30 beats per minute for a predetermined period of time. After that time, the heart rate is reduced in increments over further periods of time until the patient's mean nocturnal heart rate is reached. It is suggested that this “overdrive pacing” would alleviate the apnoeic disorders, though no clear mechanism has been elucidated explaining why this should be.

As mentioned above, these techniques are not entirely satisfactory as they are only applicable to patients whose basal heart rate is slower than normal, otherwise increasing a patients' heart rate to levels significantly above average figures for extended periods of time can be detrimental.

U.S. Pat. No. 6,126,611 also teaches increasing a patient's heart rate upon detection of the onset of an apnoea. The onset of an apnoea is detected in a preferred embodiment by detecting when a heart rate falls below a predetermined level. A pacemaker is then triggered so as to increase the patient's heart rate at the onset of the apnoea with the intention of altering the patient's sleep pattern so as to wake the patient from sleep. Waking the patient causes normal breathing to resume. The increased heart rate lasts for a predetermined period of time or until the apnoea is terminated.

This device aims to wake the patient during an apnoea. However, even in the absence of the device, an apnoea often causes a patient to waken. The usefulness of the device is therefore unclear. Additionally, by waking the patient during all apnoeas, the patient gets less sleep and therefore suffers from increased daytime somnolence.

US 2004/0216740 also describes a system for reducing central sleep apnoea. During a certain part of a patient's breathing cycle, at least a portion of the patient's exhaled breath is returned to the air supply tube. In this way, the patient's next breath contains some exhaled air and therefore an increased level of carbon dioxide. This re-breathing occurs just before or during the period of overbreathing.

The present invention seeks to alleviate one or more of the above problems.

According to a first aspect of the present invention, a device for improving the stability of a ventilation pattern of a patient comprises at least one sensor for sensing a parameter which reflects a level of lung gas in a patient and for producing an output signal indicative of said parameter, a processor adapted to receive and process the sensor output signal to assess the lung gas level, the processor being in communication with means for increasing the lung gas level of the patient, and being configured to produce a control signal for instructing said means in response to a decreasing level or predicted decreasing level of lung gas, so as to retard a decrease in said lung gas level.

According to a second aspect of the present invention, a method of improving the stability of a ventilation pattern of a patient comprises the steps of detecting a parameter which reflects a level of lung gas in a patient and causing a retardation of a decrease in said level of lung gas in response to a decreasing level or predicted decreasing level of lung gas.

According to another aspect of the present invention, a device for improving the stability of a ventilation pattern of a patient comprises at least one sensor for sensing a parameter which reflects a ventilation level of a patient and for producing an output signal indicative of said parameter, a processor adapted to receive and process the sensor output signal to assess a ventilation level, the processor being in communication with means for increasing or controlling the lung carbon dioxide level of a patient, and being configured to produce a control signal for instructing said means in response to detection of an increase in ventilation.

According to a further aspect of the invention, a method of improving the stability of a ventilation pattern of a patient comprises the steps of detecting a parameter which reflects a level of patient ventilation and retarding a decrease in the level of carbon dioxide in the patient's lungs in response to detection of an increase in ventilation.

According to another aspect of the invention, a method of improving the stability of a ventilation pattern of a patient comprises the steps of detecting a parameter which reflects a level of patient ventilation and causing the level of carbon dioxide in the patient's lungs to increase in response to a predicted deficit in the patients lungs, as a result of continuous analysis of ventilation. The predicted deficit is optionally one which would occur in the immediate future, such as within one breathing cycle.

Ventilation refers to the total volume of air taken into the lungs per unit time. It can be determined using a combination of the number of breaths per unit time and the volume of air breathing in and out during each breath. As mentioned above, patients suffering from disorders of respiratory control comprising fluctuations in ventilation tend to breath in an oscillatory pattern. A period of shallow, sometimes slow or infrequent breathing culminating in a cessation of breathing is followed by a period of more rapid, sometimes deeper breathing. Ventilation therefore oscillates, often approximately sinusoidally, or as a truncated sinusoid (truncated by the lower physical limit of ventilation during an apnoea when ventilation is zero), or in a more assymetrical pattern with a rapid rise in ventilation after an apnoea but a more gradual decline.

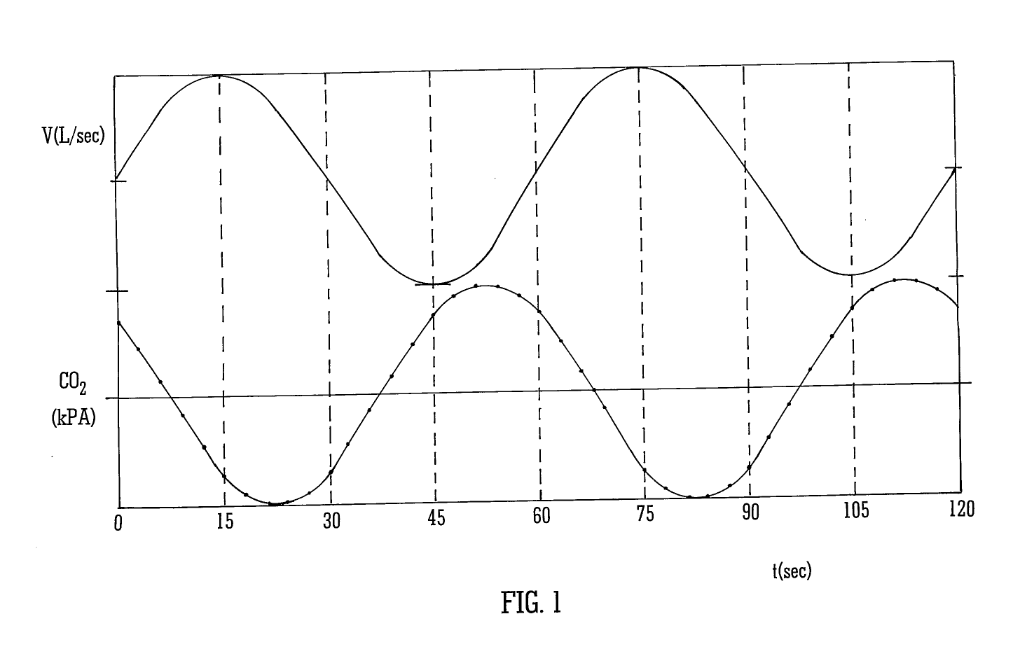

In such situations, the level of carbon dioxide in the lungs also oscillates, though not necessarily in phase with the oscillations in the ventilation. A typical ventilation pattern and corresponding lung carbon dioxide cycle is shown in FIG. 1. Ventilation (V) in litres per second and end tidal carbon dioxide (CO₂) in kPA is plotted against time (t) in seconds.

The level of oxygen in the lungs oscillates in a similar fashion to the level of carbon dioxide, though the variation in lung oxygen level generally opposes that of lung carbon dioxide. In other words, when the lung carbon dioxide level is at its peak, the lung oxygen level is at its minimum; when the lung carbon dioxide level is at its minimum, the lung oxygen level is at its maximum. Accordingly, the lung oxygen level follows a substantially equivalent pattern to that shown for the lung carbon dioxide level in FIG. 1, but is substantially 180° out of phase.

The aim of the invention is to reduce the amplitude of the carbon dioxide and oxygen oscillations and stabilise ventilation. This is done using means for increasing a level of a gas in the lungs above the level that would otherwise have been present in the absence of treatment. Treatment is applied so as to increase the level of the lung gas at a time when the level of the gas naturally present in the lungs is decreasing. The lung gas may be carbon dioxide or oxygen. Where the lung gas is carbon dioxide, the means for increasing the carbon dioxide level can be an external source of carbon dioxide, a pacemaker device operated so as to increase cardiac output, a hypoxic gas mixture or an element which adjusts the degree of the patient's respiratory airflow. Where the lung gas is oxygen, the means for increasing the oxygen can be an external hyperoxic gas mixture or a pacemaker device operated so as to reduce cardiac output. In some embodiments, the invention may use both means for increasing the carbon dioxide level and means for increasing the oxygen level when the natural levels of carbon dioxide and oxygen respectively would otherwise be decreasing.

The following discussion refers in parts to assessing a ventilation level and increasing the carbon dioxide level in the lungs above the level that would otherwise have been present when ventilation is increasing. Although ventilation levels are related to lung carbon dioxide and oxygen levels, the relationship can vary depending on the nature of the means for increasing the lung gas level and the individual patient. It is therefore advantageous to coincide the timing of treatment with the level of the lung gas rather than the level of ventilation. Accordingly, as mentioned above, in the first and second aspects of the invention, treatment is applied in response to a decreasing or predicted decreasing level of lung gas. It is to be understood that the features described below with respect to a ventilation level may be used in combination with the first and second aspects of the present invention, and so references to carbon dioxide apply equally to oxygen and references to assessing a ventilation level apply equally to assessing a carbon dioxide or oxygen level.

The system can involve causing the level of carbon dioxide in the lungs to be artificially increased above the level that would otherwise be present when ventilation is increasing. By timing the treatment to coincide with an increase in ventilation, the treatment is applied when the natural endogenous carbon dioxide level is decreasing, and so the overall level of carbon dioxide in the lungs is levelled out. In effect, a decrease in the level of carbon dioxide actually present in the lungs is retarded by the addition of carbon dioxide by treatment. Causing an increase in lung carbon dioxide levels promotes an increase in ventilation and prevents the onset of low CO₂ and an apnoea which would otherwise have followed.

References to an increase in carbon dioxide levels therefore refer to an increase above the levels that would otherwise have been present in the absence of treatment.

In a preferred embodiment, the control signal instructs the carbon dioxide increasing means such that the level of carbon dioxide is increased at a point when the rate of decrease in the natural endogenous lung carbon dioxide level is equal to or greater than a predetermined value. This has the benefit that treatment is not applied (i.e. the carbon dioxide level is not increased above natural levels) when the natural endogenous carbon dioxide level is still elevated. This can optionally be achieved by activating the carbon dioxide increasing means at a point in the cycle when the lung carbon dioxide level decreases to a threshold level. The threshold level is preferably greater than the level at which the rate of decrease in endogenous carbon dioxide is greatest. This allows for an intrinsic delay in patient response, between activating said means and actual increase in the level of lung carbon dioxide. Accordingly, the means can be activated before the lung carbon dioxide level decreases to a threshold level, which causes an increase in the level of lung carbon dioxide when or after the lung carbon dioxide level reaches the threshold.

The threshold level is preferably determined by an analysis of the sensor signal over time.

Where a patient's breathing is cyclic, the processor can be used to identify a cyclic pattern of ventilation.

Advantageously, the system is optionally able to treat irregularities in breathing without requiring them to fit a regular cyclical pattern. For example, the device could be programmed to administer carbon dioxide in concentration linearly related to the deviation of ventilation from its long-term average. This allows transient non-cyclical breathing abnormalities to be treated.

Preferably, the duration of the control signal is less than the period of the cyclic breathing. More preferably, the duration is adapted on a cycle by cycle basis to the degree of periodicity of ventilation, so that when ventilation is nearly stable only a short duration of treatment is given, and when the oscillations in ventilation are great, the treatment is given for a longer duration. In other words, the invention is adapted to deliver treatment at specific phases of the periodic breathing.

The control signal can cause the output of the carbon dioxide increasing means to follow a predetermined pattern. For example, the output of the carbon dioxide increasing means can be steady over time, such as having a square wave profile. In other embodiments, the output of the carbon dioxide increasing means can vary with time. In this way, as the level of carbon dioxide naturally present in the lungs gradually decreases, the level of carbon dioxide present in the lungs due to the intervention of the invention gradually increases, thus acting to level out the overall amount of carbon dioxide in the lungs. For example, the output of the carbon dioxide increasing means can be increased sinusoidally or in an incremental, saw-tooth pattern.

In other embodiments, the control signal can cause the output of the carbon dioxide increasing means to vary in response to real time variations in the detected ventilation. This allows more accurate treatment which is tailor made to the patient.

Preferably, the carbon dioxide increasing means has a maximum output so as to have a greatest effect on the lung carbon dioxide level when the natural endogenous level of carbon dioxide would (if untreated) be falling at its fastest rate. Matching the maximum rate of addition of exogenous carbon dioxide level with the fastest fall in the natural carbon dioxide level achieves advantageous efficiency for stabilising the patient's breathing pattern.

The output of the carbon dioxide increasing means can be seen to be a retarding force acting to retard the decrease in carbon dioxide present in the lungs.

The device can further comprise a memory unit to store the sensor output signal or a derivation thereof. This can be accessed by the processor to identify a cyclic ventilation pattern and to determine what phase in the cycle the patient is at.

The processor preferably determines the treatment to be applied by accessing at least a selection of the sensor output signal detected over a period of time, analysing the selected signal to determine the phase and amplitude of the ventilation cycle and comparing the phase and amplitude with reference data to produce an appropriate control signal. The reference data preferably indicates a suitable control signal, and therefore a suitable treatment regime, for certain phase and amplitude sets. The comparison step can involve interpolating the reference data to provide an appropriate control signal for the actual phase and amplitude.

The reference data may comprise limits in the reference phase and amplitude data, whereby the control signal is produced when the actual phase and amplitude falls between these limits.

Optionally, the device can comprise means for increasing the lung carbon dioxide level of a patient. In one embodiment, the means can comprise a source of carbon dioxide in fluid communication with a delivery device configured to deliver carbon dioxide to a patient. For example, the delivery device may be a facemask or nasal cannula. Since the carbon dioxide need not be delivered to the patient under high pressure, the delivery device need not be air-tight against the patient. Thus this embodiment of the invention is comfortable to use and therefore can achieve high compliance. This is a substantial advantage over all other forms of ventilatory treatment for stabilising disordered breathing.

Optionally, the source may be a container of carbon dioxide, such as a canister or cylinder. In this way, a selected concentration of carbon dioxide can be administered. In any case, the concentration of carbon dioxide supplied by the source is greater than the average concentration of the gas in the atmosphere.

Alternatively, the source may be a reservoir of exhaled air collected from the patient. This has the benefit of decreased cost. For additional advantage, oxygen may be added to the reservoir to prevent the occurrence of hypoxias.

Carbon dioxide from the carbon dioxide source can optionally be mixed with atmospheric air or oxygen before delivery to the patient.

Examples include delivering a gas mixture containing carbon dioxide, for example at 4, 6, 8, 10 or other percentage, with 21% oxygen and the balance nitrogen, administered from a reservoir kept near the patient, or a mixture of carbon dioxide at a predetermined concentration (e.g. 4, 6, 8, or 10%) and oxygen at a below-atmospheric concentration (e.g. 16%, 18%, or 20%).

The control signal may control an electromechanical device which adjusts the pneumatic resistance of a tube connected to the carbon dioxide source. Alternatively, the control signal may operate a valve on the source. More than one tube may be provided, each being provided with a valve or electromechanical device, so that the processor can cause different levels of carbon dioxide to be supplied by each tube. In this way, the concentration of carbon dioxide delivered to the patient can be controlled using binary logic. Preferably, this collection of tubes will include some tubes administering carbon dioxide and some administering air, and the tubes will be in a parallel arrangement. Advantageously, the resistances of the tubes administering carbon dioxide will be in ratios as follows: 1, 2 4, 8 etc, while those of the tubes administering air are in the same ratio. In this embodiment, the processor can apply complementary binary signals to the two sets of tubes, and thereby achieve a wide range of carbon dioxide concentrations whilst maintaining a constant overall resistance. A variety of alternative embodiments of resistance and switching are possible, and are well known to those skilled in the art.

In an alternative embodiment, the carbon dioxide to air ratio may be set by breathing the two gases through a tube connected to two apertures, the relative sizes of which may be adjusted electromechanically. An example of such a system would be an arrangement of closely fitting co-axial tubes, the relative orations of which can be varied by a servo-control system. The relative apertures through which gas flows between the two tubes are determined by the correspondence of holes common to the two tubes.

In an alternative embodiment of the gas administration system, the carbon dioxide is stored in a high-pressure cylinder, and administered via an electronically controllable continuously variable valve such as those commercially available from Alicat Scientific.

In another embodiment, the means for increasing the lung carbon dioxide level comprises a pacemaker. Various aspects of the pacemaker's operation can be controlled by the control signal so as to cause an increase in cardiac output. For example, the control signal may instruct the pacemaker to cause changes in the patient's heart rate, changes in the voltages outputted from the pacemaker, changes in the cardiac chamber paced or the order of their pacing, changes in a delay between pacing of chambers, changes in a delay between sensing of one site and pacing of another, or combination of these. In addition, the control signal can cause the pacemaker to deliver augmentation therapy, such as pulse trains e.g. non-excitatory stimulation, cardiac contractility modulation or post-extra systolic potentiation therapy.

The increased cardiac output will result in a rise in the rate of return of carbon dioxide rich blood to the lung reservoir. This in turn can (via the chemoreflex) influence ventilation.

The treatment can be varied in terms of duration of treatment as well as magnitude of treatment. For example, the amount by which the heart rate is increased can be varied and/or the flow of carbon dioxide to the patient can be varied and/or the concentration of carbon dioxide delivered to the patient can be varied.

The sensor is one which can sense a physiological variable which varies with ventilation and so reflects the ventilation level. The sensor can be one or more of a ventilatory sensor, a heart rate monitor, a blood velocity, heart rate or thoracic impedance monitor, a respiratory strain gauge, a blood carbon dioxide, oxygen, lactate or pH level sensor, an expired carbon dioxide or oxygen sensor, a thermistor or a peripheral oxygen saturation monitor, a movement sensor such as a piezo electric crystal or an accelerometer, or other suitable sensor, or a combination thereof.

Examples of sensors are discussed in U.S. Pat. No. 5,540,773 and U.S. Pat. No. 6,132,384 which describe a system for measuring respiratory effort by monitoring airway pressures, and U.S. Pat. No. 5,174,287 which describes a system for monitoring electrical activity associated with contractions of the diaphragm and the pressure within the thorax and upper airway.

In still a further embodiment, the means for increasing the lung carbon dioxide level may comprise both a source of carbon dioxide and a pacemaker, as described above. This provides flexibility in the way in which treatment is applied.

The components of the device may be integral with some or all of the other components, connected to some or all of the other components, for example by electrical wires, fibre optic communication, or may wirelessly communicate with some or all of the other components, for example by way of infra-red data transfer or electromagnetic transmission, such as that achievable with a telemetry head. For example, one or more sensors may be integral with a pacemaker. The processor my also, in some embodiments, be integral with the pacemaker.

In one embodiment of the invention, the processor can be used to measure chemoreflex gain and delay from an analysis of carbon dioxide and ventilation signals. This can be done by introducing transient afferent stimuli and detecting the downstream effect on ventilation. The stimuli can be repeated frequently and at variable intervals so as to calculate an averaged response from which a time can be calculated between stimulus and response.

Optionally, the device can comprise one or more sensors to detect a patient physical activity level and/or degree of wakefulness. The device can therefore have an operating mode in which it would cause treatment only when a patient is at rest or is asleep, preferably having been in that state for a pre-determined period of time.

As discussed above, in preferred aspects of the invention, a lung gas level is assessed and means for increasing the lung gas level can be activated in response to a decreasing or predicted decreasing level of the lung gas, so as to retard a decrease in said lung gas level. The lung gas may be carbon dioxide or oxygen. The principles of these aspects of the invention are substantially the same as described above. Namely, by causing an increase in carbon dioxide or oxygen at a specific phase of a breathing pattern to balance out a decrease in the level of carbon dioxide or oxygen naturally occurring in the lungs, an apnoea that would otherwise have resulted is avoided.

Accordingly, embodiments of the invention which retard a decrease in lung carbon dioxide level can be combined with any of the features described above, including the use of an external carbon dioxide source and/or a pacemaker operated to cause an increase in cardiac output. Additionally, the means for increasing the level of lung carbon dioxide can include a source of hypoxic gas, i.e. a source of gas in which the oxygen content is below the oxygen content of atmospheric air. The hypoxic gas mixture may comprise 16%, 18% or 20% oxygen, most or all of the balance of the gas being made up of nitrogen. Supplying a hypoxic gas mixture to a patient in response to a decreasing or predicted decreasing level of lung carbon dioxide stimulates ventilation, causing a retardation of the decrease in the level of lung carbon dioxide. Further, means for increasing the level of lung carbon dioxide could include an airflow control element which acts to adjust, for example reduce, the degree of the patient's respiratory airflow. The airflow control element can manipulate ventilation by interfering with the body's natural ventilatory efforts. For example, there may be provided a physical restraint adapted to alter the volume of breaths taken by the patient. The physical restraint can restrain the movement of the patient's chest and/or abdomen to varying degrees to control the volume of breath that can be taken in. For example, an elastic vest-like device which can be tightened around the chest and/or abdomen to varying degrees can be used. Alternatively or in addition, there may be provide a source of exhaled air collected from the patient as described above. In this example, there may be provided a pair of conduits, one of which leads to the atmosphere and the other of which leads to the source of exhaled air, such as a rebreathing bag. There may be valves to vary the balance of respiratory airflow through the two conduits.

An advantage of restricting ventilation in this way is that a close-fitting facemask is not required, and so patient comfort and therefore compliance is improved.

Embodiments of the invention which retard a decrease in lung oxygen level can also be combined with the features discussed above, including those which are adapted for use with a gas source for delivering gas to a patient, the difference being that the gas source comprises oxygen, specifically a hyperoxic gas mixture. However, since the level of natural oxygen in the lungs varies generally inversely with the level of natural carbon dioxide, treatment by delivering oxygen is applied half a phase after (or before) treatment by carbon dioxide delivery i.e. when the level of oxygen in the lungs would otherwise be decreasing. The description above of the device and method used to increase carbon dioxide levels in the lungs using a gas source is therefore fully applicable to the aspects of the invention which increase oxygen levels in the lungs, except that timing of the treatment is half a phase different. In this embodiment, a hyperoxic gas mixture, such as 25%, 40%, 60% or 100% oxygen, with the balance or most of the balance being nitrogen, may be delivered to the patient.

Alternatively, or in addition, the means for increasing the lung oxygen level can comprise a pacemaker device, the control signal being adapted to instruct the pacemaker to cause a reduced cardiac output in response to a decreasing or predicted decreasing level of oxygen in the lungs. Reducing cardiac output has the effect of reducing the return of carbon dioxide to the lungs which in turn retards the decrease in oxygen levels. The reduction in cardiac output is timed so as to have its effect on the lung oxygen level when the lung oxygen level would otherwise be decreasing.

Embodiments of the present invention will now be described, by way of example only, with reference to the following drawings in which:

FIG. 1 shows the variation in ventilation and carbon dioxide levels for a typical patient;

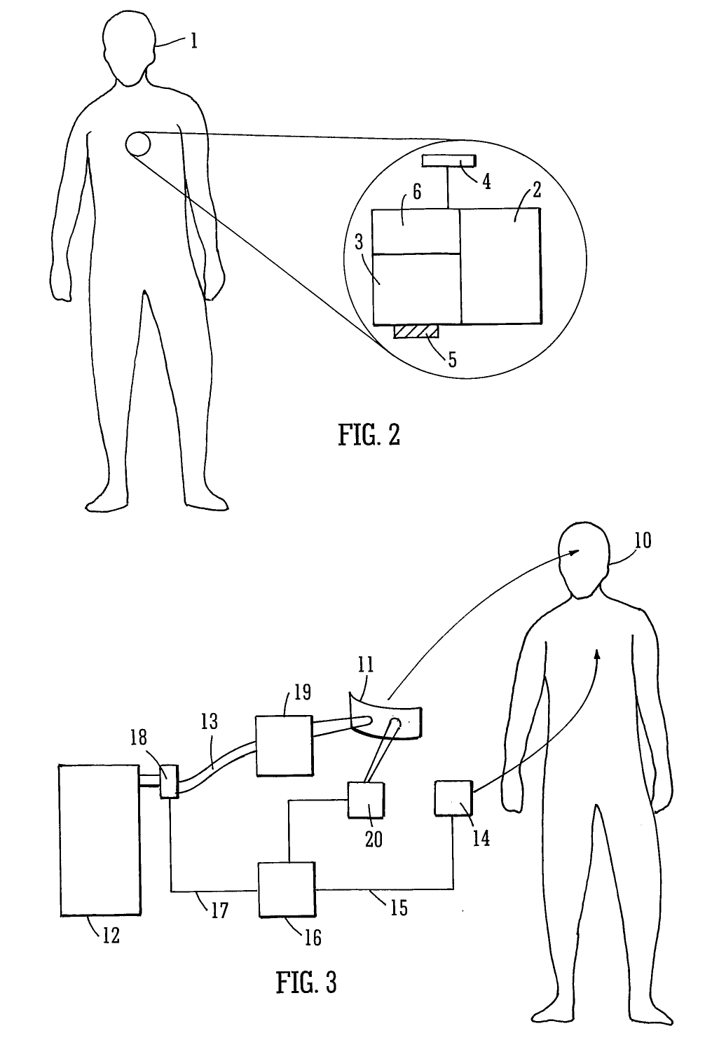

FIG. 2 shows a schematic diagram of the device of one embodiment of the invention;

FIG. 3 shows a schematic diagram of the device of another embodiment of the invention;

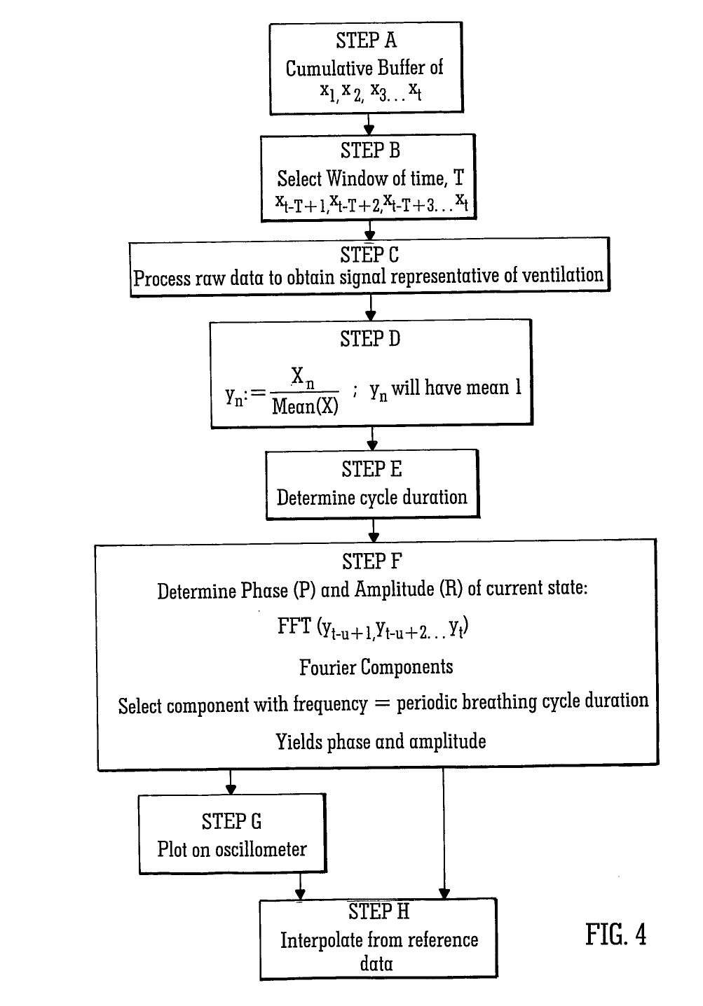

FIG. 4 shows a flow chart for determining treatment timing and dose;

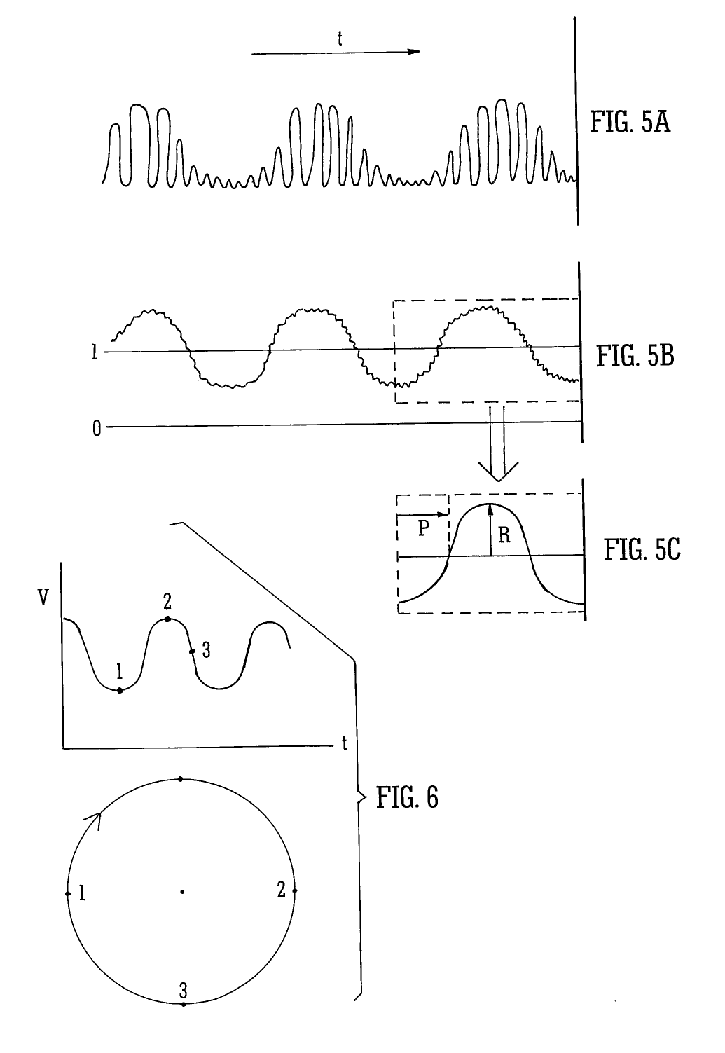

FIGS. 5a, 5b and 5c show data collected from a respiratory strain gauge before and after processing;

FIG. 6 shows the relationship between data points on a curve showing ventilation against time and points on an oscillometer chart;

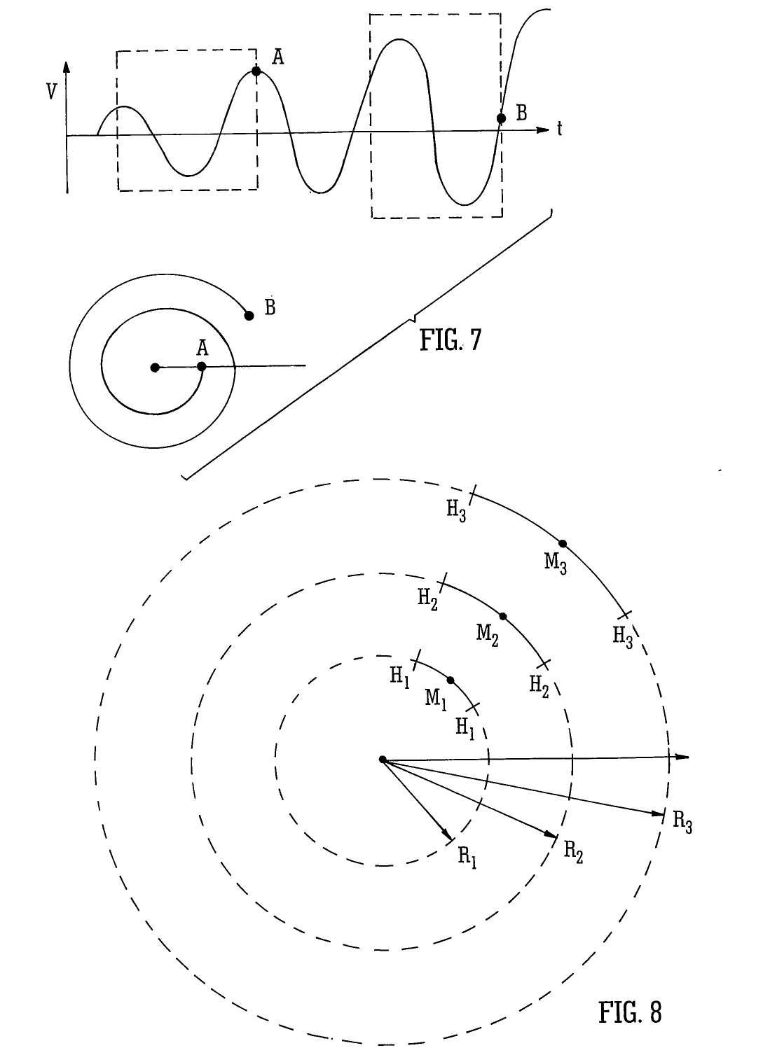

FIG. 7 shows an oscillometer chart for a deteriorating pattern of breathing;

FIG. 8 shows a sample reference oscillometer chart;

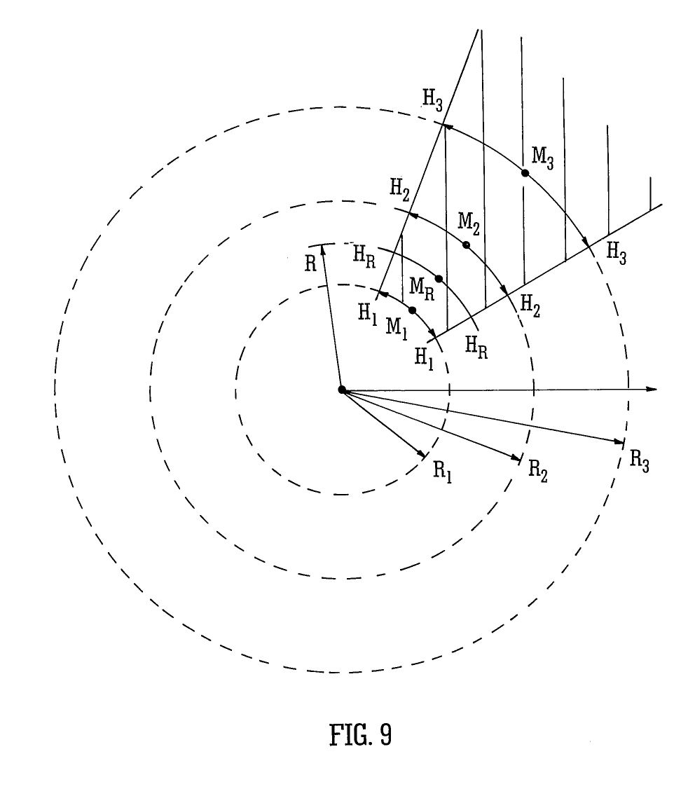

FIG. 9 shows the interpolation of a treatment level from a reference oscillometer chart;

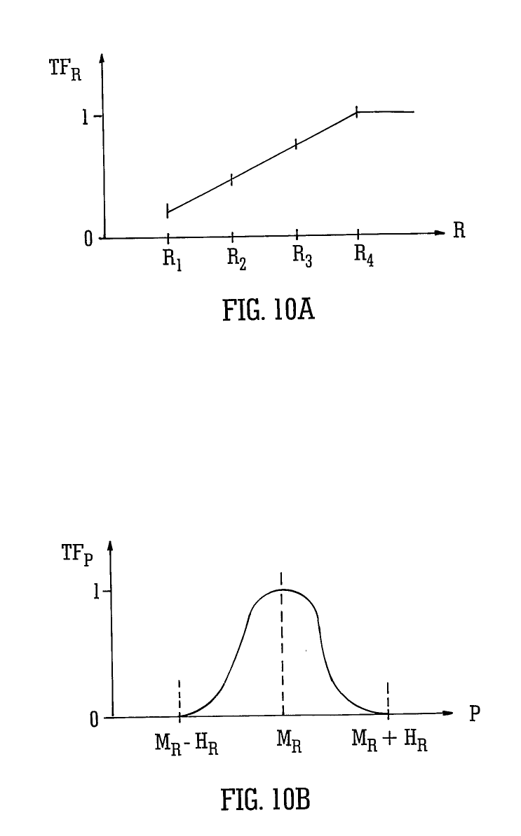

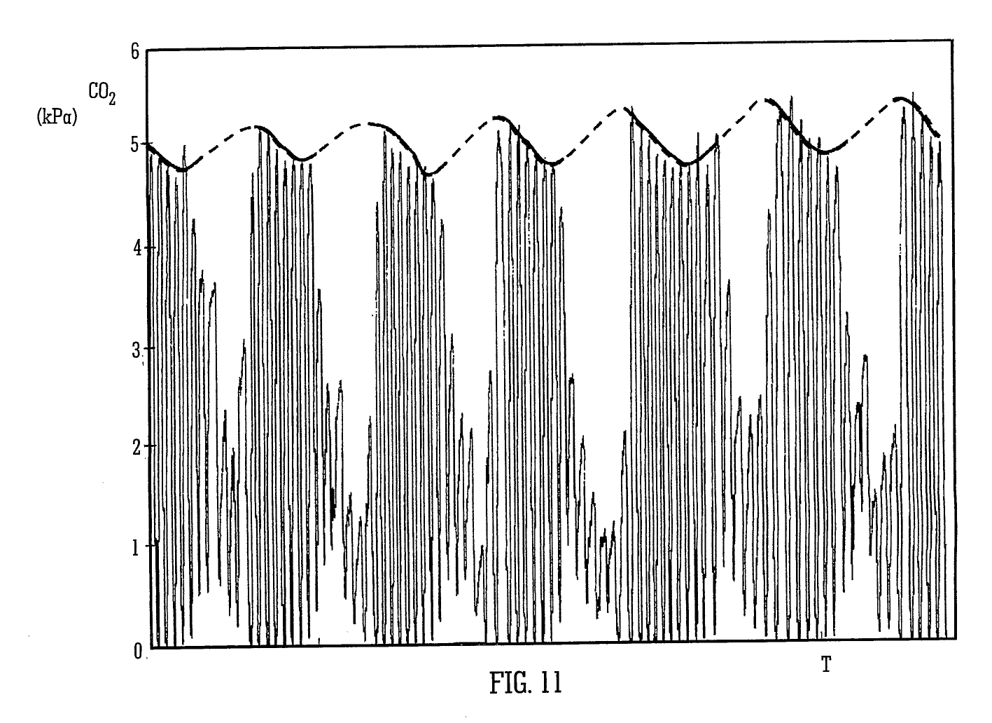

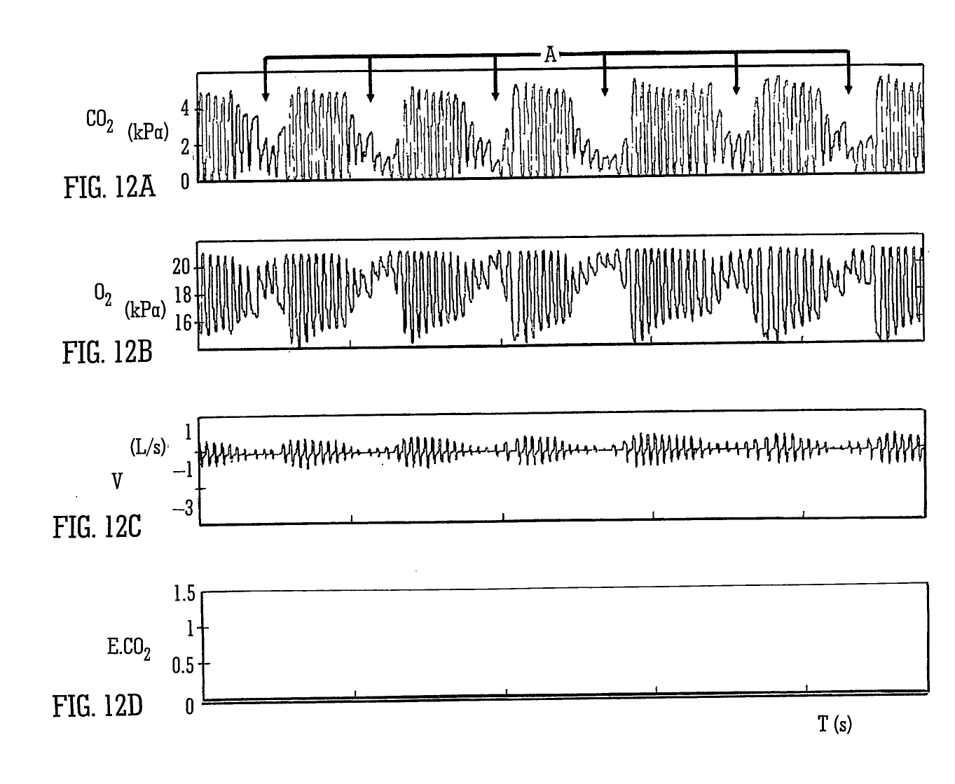

FIGS. 10a and 10b show examples of therapy factors

FIGS. 11 to 14 show charts reflecting a patient's ventilation, carbon dioxide and oxygen levels before and during treatment with the present invention

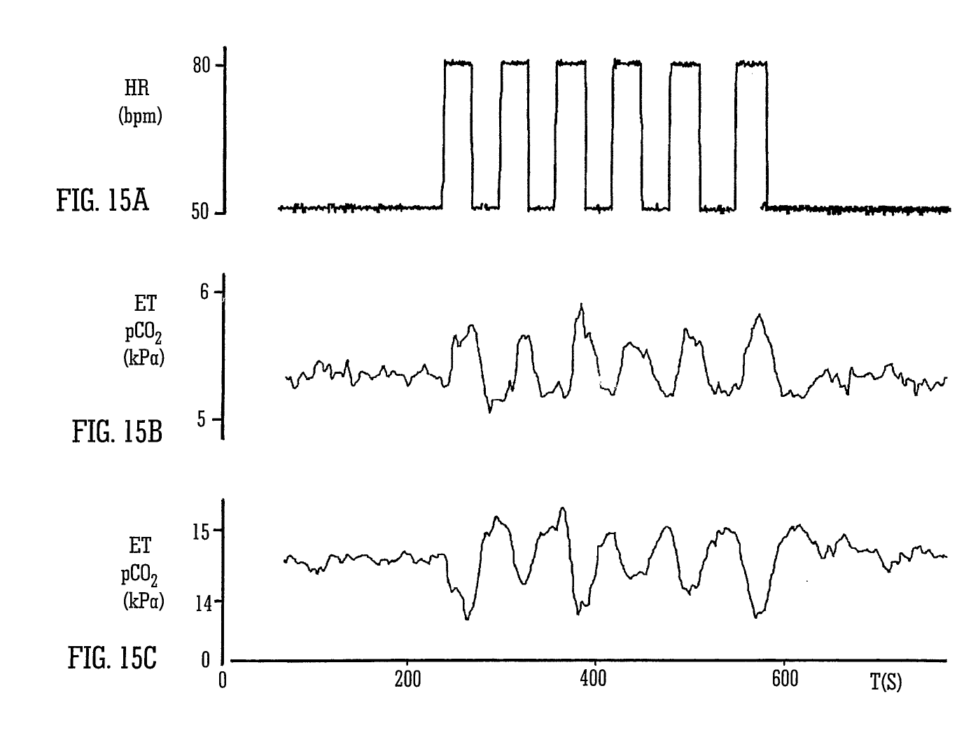

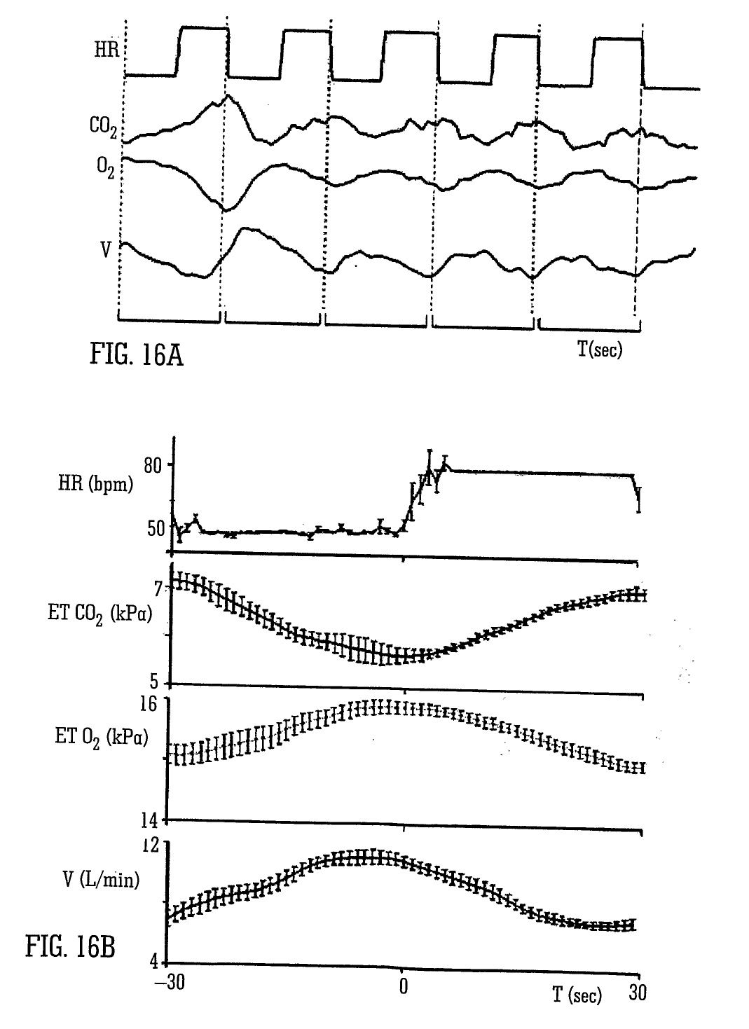

FIGS. 15 and 16 show the effect on ventilation, carbon dioxide and oxygen levels of a patient by increasing cardiac output using a pacemaker.

In a first embodiment of the present invention, shown in FIG. 2, a device comprises a pacemaker 2 which can be implanted in a patient 1, a sensor 4 which can collect physiological data related to the patient's breathing and/or cardiovascular function and a processor 3 to control the operation of the device. The pacemaker 2 is of known type, such as the Medtronic Insync III pacemaker. The sensor 4 could be a transthoracic impedance sensor which senses the resistance to flow of an electrical current across the lungs, and therefore yields an index of lung volume, although other sensors for detecting a physiological variable which reflects ventilation could be used. The sensor is able to communicate with the processor 3 which, in this embodiment, is built in to the pacemaker device. The pacemaker device, incorporating the pacemaker 2 and processor 3, and sensor 4 can be implanted in the patient 1. The processor 3 could be adjusted manually via an external telemetry head 5 (e.g. for changing programming parameters).

The signal from the sensor 4 is indicative of lung volume and therefore reflects a patient's ventilation and the lung carbon dioxide and oxygen levels. The signal is transmitted to the processor 3 and is stored in a memory 6 located with the processor 3. The processor 3 accesses the stored information to assess the ventilation, lung carbon dioxide and/or oxygen levels and its variation over time. The processor 3 therefore determines whether a cyclic pattern is present and thus, whether cyclic breathing is occurring. The processor 3 can therefore identify apnoeas and hyperpnoeas in a cyclic pattern.

After an apnoea, the patient's ventilation increases and this is detected by the processor 3. The processor then produces a control signal to operate the pacemaker 2 so as to increase the heart rate of the patient 1. The control signal can also alter other pacing parameters instead of, or in addition to, increasing the heart rate. For example, the pacemaker 2 can change the cardiac chamber paced or the delay between the chambers paced. This changes the cardiac output and as a result, the level of carbon dioxide in the patient's lungs increases. The control signal operates the pacemaker 2 such that lung carbon dioxide level is increased at a time when the natural carbon dioxide level is decreasing to a minimum. This burst of induced carbon dioxide prevents the subsequent decrease in ventilation which would otherwise follow as a result of the low natural lung carbon dioxide level.

The pacemaker 2 is activated in this way only for a short time, which is less than the period of the periodic breathing. More specifically, the elevation in cardiac output is engendered by the pacemaker 2 during the part of the ventilatory cycle which would, in the untreated state, have the greatest rate of fall in lung CO₂.

The device uses the control signal to cause the pacemaker parameters to change. This in turn causes the cardiac output to change and carbon dioxide levels to alter.

In a second embodiment, shown in FIG. 3, the carbon dioxide level in the lungs is increased by delivering carbon dioxide to the patient's ventilation system via a face mask 11, or similar delivery device. The carbon dioxide is stored in a gas canister 12 which is in communication with the face mask 11 via a tube 13. A respiratory strain gauge 14, or other type of sensor such as a thermistor, flowmeter, pneumotachograph, pulse oximeter, is used to detect a parameter reflective of the patient's breathing (chest wall movement in the case of the strain gauge). The sensor 14 is connected via electrical wires 15 or other suitable communication means to a processor 16 which can be positioned with the gas canister 12, or remote therefrom as shown in FIG. 3. The processor 16 sends the control signal via electrical wires 17 or other suitable communication means, so as to control the carbon dioxide source.

The patient's breathing pattern is identified in the same way as described above with respect to the pacemaker device. Specifically, the signal from the sensor 14 over a period of time is collected and stored in a memory. The stored information is then analysed by the processor 16 so that the variation in ventilation level is determined. At the appropriate time in the periodic breathing cycle, the processor 16 produces a control signal which causes gas from the carbon dioxide canister 12 to be delivered to the patient 10 via the face mask 11 and thus to the patient's lungs. In one embodiment, this appropriate time can be defined simply as the time at which the ventilation rises above a certain threshold—for example the mean. In a preferred embodiment this appropriate time is determined by an automatic analysis of the time course of ventilation in the recent past using a series of steps such as those we describe below, with respect to FIG. 4. The advantage gained from this preferable embodiment is that, if the means of delivering carbon dioxide necessarily incorporates a delay before the carbon dioxide arrives in the lung, the time at which the control signal is delivered to the means of delivery can be programmed to be earlier. The timing can be earlier to any desired degree, even if the desired degree exceeds half a cycle, which would mean the carbon dioxide administration control signal is activated while carbon dioxide is still rising. The rise in exogenous carbon dioxide therefore circumvents the reduction in endogenous carbon dioxide. The control signal operates an electromechanical device 18, such as a solenoid or balloon valve, which adjusts the pneumatic resistance of the tube 13 connected to the carbon dioxide canister 12. This tube 13 opens into a reservoir 19 held at or near atmospheric pressure coupled to the face mask 11. Another electromechanical device 20 controls the inflow of room air at atmospheric pressure, which is also coupled to the face mask. The electromechanical devices 18,20 therefore adjust the relative proportions of air and carbon dioxide inhaled by the patient, and can accurately deliver variable concentrations of carbon dioxide.

As an alternative, the control signal may electromechanically operate a valve on the gas canister to change the proportion of atmospheric or pressurised air and carbon dioxide applied from a pressurised cylinder, with or without an intermediate low pressure reservoir.

In a variation (not shown), a number of parallel tubes are provided with resistances of ratios of powers of two such that control of resistance maybe effected using binary logic. Another implementation involves incorporating rotary gas regulator valves operated by an electromechanical servo control system.

The device has built in safety mechanisms whereby continuous delivery of carbon dioxide enriched air is prevented for longer than a predetermined period. These can be incorporated into the processor 3,16, or preferably be provided by an additional separate control system making it independent from failure of the main processor. Further, in the event of electrical or controller failure, the device is provided with a safety system to switch to respiration of normal atmospheric air until it is manually reset.

A further embodiment is similar to that described above using a carbon dioxide canister. However, the facemask collects exhaled air and feeds this to a reservoir, thereby storing exhaled, carbon dioxide rich, air. When the processor determines that treatment is necessary, the control signal causes exhaled air to be delivered to the face mask in the same way as that described above for delivering carbon dioxide from a canister.

In other embodiments of the invention, the signal from the sensor is processed by the processor to assess the lung carbon dioxide and/or oxygen level. The processor determine whether there is a cyclic pattern in the variation of the lung gas levels and can therefore identify apnoeas and hyperpnoeas as discussed above. The processor produces a control signal in response to a decreasing lung gas level or predicted decreasing lung gas level which operates lung gas level increasing means so as to retard the decrease in the lung gas level. The carbon dioxide increasing means can be the pacemaker, carbon dioxide gas canister or the reservoir of exhaled air as discussed above. Alternatively, it may be a hypoxic gas mixture comprising 16%, 18% or 20% oxygen. The oxygen increasing means can be a hyperoxic gas mixture comprising 25%, 40%, 60% or 100% oxygen supplied to the patient in the fashion described above with regard to the carbon dioxide gas supply. Alternatively, a pacemaker can be used to reduce a patient's cardiac output. The pacemaker can be as described above, and can be operated to reduce the patient's heart rate and/or alter other pacing parameters which reduce cardiac output at a time when the lung oxygen levels are decreasing.

In some examples, both carbon dioxide and oxygen increasing means are used in response to a decrease or predicted decrease in the level of carbon dioxide and oxygen respectively. One or more of the above-mentioned means may be used in combination.

In the above examples, the processor causes the level of carbon dioxide or oxygen to increase in the lungs when the natural endogenous level is decreasing to a minimum. Since there is an intrinsic delay between the application of treatment, such as an increase in heart rate, and the resultant increase in carbon dioxide or oxygen levels in the lungs, it is preferable to initiate the treatment at a time before it is desired to increase the carbon dioxide or oxygen level in the lungs. The processor may be programmed to activate the carbon dioxide or oxygen increasing means at a predetermined time before the increase is desired, and this predetermined time can be based on known, typical delays.

However, since different patients react in different ways to the treatment, particularly with different speeds, it is preferable that the device can learn the intrinsic delay of a particular patient so that treatment can be initiated at the optimum time before the carbon dioxide or oxygen increase is desired.

The following is a discussion of how the optimum timing of treatment can be assessed when carbon dioxide increasing means are used. Naturally, the same procedure can be used for systems using oxygen increasing means, the procedure being modified so as to commence treatment at the appropriate time in the cycle of varying oxygen level.

A cycle of repeated doses of test treatment is delivered, such as one dose per minute or a frequency to match the frequency of the patient's spontaneous cyclic breathing. After a few cycles of test treatment, the pattern of breathing will become periodic, or will have been brought into phase with the test treatment. The device can therefore determine the delay between the onset of a test dose and the peak in the signal reflective of ventilation.

The way in which the devices described above determine when treatment is necessary and the magnitude of treatment to be applied will now be described with reference to FIGS. 4 to 10. Referring firstly to FIG. 4, in Step A, sensor signals x₁, x₂, x₃ . . . xₜ are collected over a period of time, such as a number of seconds, t, and stored in the memory. The sensor senses a parameter which varies with ventilation, and so these sensor signals represent a variable which reflects ventilation and which therefore oscillates with the cyclic breathing pattern, though perhaps not in phase. The samples should therefore be taken at a frequency much faster than the period of the periodic breathing.

Certain stored sensor signals (x_{t-T+1}, x_{t-T+2}, x_{t-T+3} . . . xₜ) gathered during a window of time, T, are retrieved from the memory (Step B). For example, FIG. 5a represents raw data collected over a period of time from a respiratory strain gauge. As an option, the period of time, T, covered by the signals is determined by applying a Fourier transform (or other signal analysis method providing similar information, known to the skilled technician) to the signals gathered over another, longer period of time so as to determine the period of the periodic breathing. The window duration, T, is then set to this period or a multiple thereof.

As mentioned, measurements of ventilation can be obtained from any of several sources and each source has an appropriate form of processing in order to obtain a useable ventilation signal in order to detect the instantaneous amplitude and phase of the cyclic breathing cycle. This processing is carried out in Step C.

For example, if the signal is from a flow meter with positive voltages representing inspiration and negative representing expiration, an appropriate initial sequence of steps would be to rectify the signal (make all the negative values positive), and then apply a low pass filter for example a Hanning window or another of the many known to those skilled in the art. This will provide a signal which does not show individual breaths as oscillations, but rather smoothes these out and shows only the fluctuations in respiration corresponding to the periodic breathing cycle.

If the signal is instead from a sensor that detects chest wall position (rather than rate of movement), such as the signal shown in FIG. 5a, then an appropriate process of steps is to begin with a differentiation step (calculating the differences between successive voltages from the sensor). This generates a signal equivalent in principle to that which may be obtained from a flow meter and can then undergo the above steps of rectification and low pass filtering.

If the sensor produces a signal that is non-linearly related to ventilation, it would be advantageous to include a step in the processing that applies a calibration curve to generate a signal that is linearly related thereto.

Advantageously for the subsequent steps in the analysis that may be dealt with by digital processing, the data could now be normalised (Step D). The data is scaled in relation to the long time average of ventilation such that a patient with persistently stable ventilation will persistently have a ventilation value of 1.0, whereas if a patient had an episode of apnoea, during the apnoea ventilation would be scored as zero, and during hyperpnoeas the ventilation would be scored with values greater than 1.0, and all ventilations between these extremes would be scored on this scale. The simplest means of normalisation is to divide the raw values reflecting ventilation (stored in the cumulative buffer) by their mean value in order to obtain a normalised set of data (yₙ).

FIG. 5b shows the result of differentiation, rectification, low pass filtration and normalisation on the raw data of FIG. 5a.

Two windows of time are now defined automatically by the processor in the preferred embodiment. The first window, T, as discussed above, is a long window of time (for example 10 or 15 minutes). Over the normalised ventilation data stored in this long window, it is possible to establish what is the duration of the periodic breathing cycle by any one of a large number of automatic means readily known to those skilled in the art (Step E). For example, this could be done by counting of the peaks or troughs, counting the zero crossings or advantageously by applying a Fourier transform, and selecting the dominant frequency within a range of plausible values for the cycle time of periodic breathing. Such a band of plausible values may run, for example, between 45 seconds and 90 seconds.

The second window is a short window, U, intended for processing to determine the precise phase and amplitude of the current cycle of periodic breathing. Advantageously the processor will take the value of the periodic breathing cycle length taken from analysis of the long window, T, and use it as the definition of the length of the short window, U, (Step F). This short window contains only information pertaining to the current cycle (for example, about the past 1 minute) which gives the advantage that it is very responsive to treatment.

The amplitude (R) and phase (P) of the periodic breathing is then calculated automatically. This process begins with Fourier analysis on the normalised data within the short window (for example from 1 minute ago to the present) to obtain the Fourier components. The component with the frequency which matches the duration of the periodic breathing cycle is then selected automatically, and its amplitude and phase determined to yield R and P. For example, for the raw data of FIG. 5a, FIG. 5c shows the component of the oscillation in ventilation at the frequency of periodic breathing, isolated and quantified by Fourier analysis, to give an amplitude R and phase P. Advantageously the full Fourier analysis may not be calculated since the frequency of interest is known in advance. Instead, the usual iterative process to calculate Fourier components of different frequencies (well known to those skilled in the art) can be replaced by a single step calculating only the amplitude and phase of interest.

A representation can then be built up of how the ventilation cycle varies over time. A convenient representation can be obtained by plotting the data on an oscillometer (Step G). It is to be understood that actually plotting the data on an oscillometer is not essential to the operation of the device, and is used here merely to provide a medium of understanding the manipulation of the data. The way in which the points on a curve showing ventilation (V) against time (t) correspond to those on an oscillometer is shown in FIG. 6. The angular position of a point on the oscillometer chart indicates the phase of a signal at a given time and the distance from the centre of the chart indicates the maximum amplitude of the cycle. Point 1 corresponds to the trough in ventilation, point 2 to the peak and point 3 to a mid-point in ventilation. The plot of normalised ventilation (V) against time (t) shown in FIG. 7 represents a gradually deteriorating pattern of breathing and can be plotted on an oscillometer chart as shown. An increasing amplitude of the periodic breathing pattern is represented on the chart by a spiral, the radius of the spiral increasing as the oscillations in ventilation gradually increase in amplitude.

The device is therefore able at any point in time to determine at what phase in the pattern of periodic breathing a patient is at, and what the deviation from average ventilation is. By comparing the current point on the chart (i.e. the current phase and amplitude of periodic breathing) with look up tables that describe the degree of therapy at given regions of an oscillometer chart, the processor can produce the appropriate control signal so as to administer the appropriate treatment.

A sample reference chart is shown in FIG. 8. The chart has a number of reference radii, in this case three reference radii (R₁, R₂, R₃), which correspond to three different amplitudes of periodic breathing. On each reference radius, a treatment region corresponding to a short section of the cycle is marked out. The treatment region is centred around a mid-point on the reference radius (M₁, M₂ and M₃) and extends for a half width H₁, H₂, H₃ on either side of the point. This region represents the phase in the breathing cycle during which treatment should be applied to the patient. It is seen that the greater the amplitude of the periodic breathing (corresponding to radius R₃), the longer the treatment is to be applied. This is because the periodic breathing is more severe and therefore can be treated more vigorously.

The device can be programmed with the reference oscillometer charts before the device is put to use with a patient, the timing and extent of treatment for points on the reference chart being determined based on known standard cardio-respiratory responses or analytical theory derived from mathematical models of the interaction between gas exchange, ventilation, gas transport and the cardiovascular system. Alternatively, the reference data may be obtained by the patient undergoing a session of monitoring of responses to a series of stimuli (eg the response times and magnitude of responses to a given exogenous CO2 concentration, or the endogenous CO2 increment obtained following a given change in pacemaker parameters).

Naturally, a patient's actual breathing pattern may not follow the reference radii exactly. Advantageously therefore, a treatment region for an actual breathing pattern can be interpolated from the reference data, for example using linear interpolation. Referring to FIG. 9, the mid-point M_{R} and half width H_{R} for a periodic breathing pattern following the path at radius R can be calculated by any known interpolation algorithm—for example the following:

$${M}_{R} = {M}_{1} + \frac{( R - {R}_{1} )}{( {R}_{2} - {R}_{1} )} \times ( {M}_{2} - {M}_{1} )$$

$${H}_{R} = {H}_{1} + \frac{( R - {R}_{1} )}{( {R}_{2} - {R}_{1} )} \times ( {H}_{2} - {H}_{1} )$$

At amplitudes less than the smallest reference radius, the processor can determine that no therapy is necessary, since the periodic breathing is not severe. At amplitudes greater than the largest reference radius, the processor may apply treatment as if the amplitude corresponds to the largest reference radius.

Once the processor has calculated the treatment region of a given pattern of breathing, the processor determines whether the current phase of breathing is within the treatment region (i.e. within M_{R}+/−H_{R}). If the processor finds that the current phase is anywhere within the treatment region, then the processor produces a control signal to apply some form of treatment is applied.

It can be seen that treatment should be applied when the amplitude and phase of the current signal falls within the wedge-shaped region in FIG. 9. The definition of the wedge-shaped region can be stored in the device, for example, in a table of values of R, M, and H, which delineate its outer boundaries.

In preferred embodiments, the processor can vary the magnitude of treatment to be applied, such that a greater level of treatment is applied at times when the natural endogenous carbon dioxide level would be expected to be falling most rapidly, as well as when the processor detects a large amplitude corresponding to severe periodic breathing. The processor can dictate the magnitude of treatment according to the following regime:

T=Tₘₐₓ×TF_{R}×TF_{P}

where T is the level of therapy to be applied; Tₘₐₓ is a master control variable which determines the maximal level of therapy that the system is currently prepared to deliver; TF_{R} is the therapy factor due to the radius corresponding to the current signal of that breathing pattern; and TF_{P} is the therapy factor due to the phase angle of the current signal. TF_{R} and TF_{P} can each have a value between 0 and 1. The value of T therefore can vary between zero (no treatment) and Tₘₐₓ which is maximal therapy.

The therapy factors can vary linearly across the range of radii or phases, such as in FIG. 10a. However, it is preferred that TF_{P} follows an inverted cosine profile across the range of phase angles, as seen in FIG. 10b. This gives the advantage that the profile of CO₂ administered over time resembles the profile of the deficit in CO₂ that would occur if treatment was not administered.

To avoid applying too much treatment, the value for the maximum level of therapy for a particular breathing pattern can initially be set to 0, but be increased gradually with time, such as every few minutes, until the breathing pattern has been stabilised satisfactorily, and reduced again if the treatment seems to be overtreating and worsening the periodic breathing.

In this way, the level of treatment is calculated in real time in response to the patient's breathing at that time.

Advantageously, the system will intermittently switch off interventions for a predetermined duration (advantageously one or more cycle lengths of periodic breathing, as determined by the analysis of the long window), for the purpose of being able to detect the incremental benefit of the therapy on respiratory control stability. Thus the system is able to detect the unlikely but conceivable circumstance in which respiratory control stability is made worse by therapy, and disable therapy until manual intervention takes place at a subsequent convenient opportunity.

It is to be noted that although throughout this document, for simplicity, the processor is described as detecting an “increase in ventilation” as the trigger for administration of therapy, in reality, by the method and apparatus herein described, the processor is determining the phase within the periodic breathing cycle using the ventilation data of one or more cycles of periodic breathing to make the determination. As a result it is capable not merely of detecting a rise in ventilation once it has happened, but also of automatically predicting a rise in the near future. Moreover it can predict, with equal facility, any predetermined phase within the periodic breathing cycle, such as peak, trough, or any desired intervening time point. Thus the therapy algorithm could equally easily be described in terms of detecting and preventing declines in ventilation: the apparatus and method would be generally the same.

Advantageously, the system has the ability to adjust therapy to fit timing requirements of the individual patient's breathing cycle i.e. to take account of the intrinsic delay between treatment and carbon dioxide increase. This could be achieved using a system of automatic assessment of efficacy of therapy. In one embodiment, the efficacy of administering therapy at an initial, predetermined, phase is tested by automatically performing a small trial of treatment versus no treatment and comparing the size of the oscillations obtained. For example, a period of five minutes in which no treatment is applied can be followed by a period of five minutes in which maximal therapy is applied 0.25 cycle before the peak of ventilation, followed by five minutes of no treatment and a further five minutes of treatment. The amplitude of oscillation of ventilation (expressed as a proportion of mean ventilation, i.e. using the normalised values described) can be averaged for the treatment episodes, and separately averaged for the no-treatment episodes. Advantageously the segment of time used for measurement of these amplitudes would not include an initial period (for example 1, 2 or 3 minutes) but rather would be the latter part of the episode. The difference between the oscillation amplitude in the treatment episode and the non treatment episode is a measure of efficacy of treatment (with negative values indicating beneficial treatment). During the night a series of slight modifications to the timing of treatment are also tried and their benefits quantified. In the example given, these might be to administer maximal carbon dioxide at 0.20 cycle or 0.30 cycle before peak of ventilation. If the modified treatments yield greater benefits than the current treatment regime, the treatment regime is changed to the improved value.

Advantageously, the range of these timings can be restricted to those that are physiologically plausible.

Advantageously, the data is cumulated over more than one night, so that a large body of data is available for selection of the best therapy timing.

Advantageously, the relationship between the timing of treatment and the effectiveness of treatment is modelled by the system by fitting to a curve, for example using least squares regression to a quadratic formula (by an algorithm familiar to those skilled in the art) so that the most effective treatment timing can be calculated. By using a bank of data from recent days (which is continually updated with removal of data from the distant past, and addition of new data) this optimum treatment time is kept updated to fit the patient's condition which may change gradually with the passage of time.

In alternative, simpler, embodiments, the processor detects when to commence treatment, but carries out treatment according to a pre-programmed regime. For example, the control signal can cause the level of treatment to follow a square, sinusoidal or saw-tooth profile. For example, a pacemaker can be caused to increase the heart rate by 10 beats per minute for a period of time, or increase the heart rate by 2 beats per minute in five steps to reach a maximum increase of 10 beats per minute before decreasing the heart rate in similar increments. These profiles can be pre-programmed based on known, standard treatment regimes or can be tailor made to the patient.

As discussed above, in preferred aspects of the invention, a lung gas level is assessed and means for increasing the lung gas level can be activated in response to a decreasing or predicted decreasing level of the lung gas, so as to retard a decrease in said lung gas level. The principles of these aspects of the invention are substantially the same as described above. Accordingly, the system described above for determining the appropriate timing of the treatment can be readily applied to, and combined with, the first and second aspects of the invention.

To demonstrate the effectiveness of the present invention, FIGS. 11 to 14 show the results of supplying carbon dioxide to a patient demonstrating periodic breathing. The patient's unaltered breathing pattern is recorded in FIG. 11. The amount of carbon dioxide at the patient's mouth is plotted against time (T), which gives an indication of individual breaths. The end-tidal carbon dioxide is plotted in broken lines, showing the fluctuation of the patient's lung carbon dioxide level. It can be seen that the carbon dioxide level measured at the mouth does not directly reflect end-tidal carbon dioxide, since the troughs in the mouth measurements do not coincide with the troughs in the end-tidal levels. This is because, during an apnoea, the patient breathes out only gas from their dead-space and not gas from the lungs.

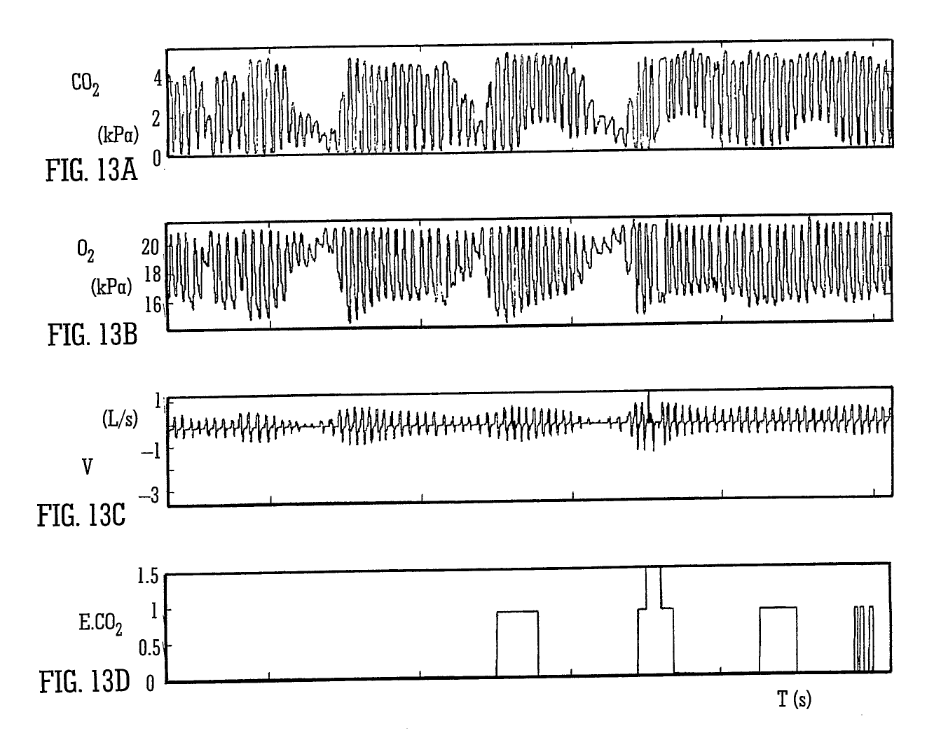

FIGS. 12a to d show the relationship between carbon dioxide, oxygen and ventilation levels. FIGS. 12a and 12b show measurements of mouth carbon dioxide and oxygen respectively against time. FIG. 12a is therefore a similar chart to that shown in FIG. 11. FIG. 12c shows ventilation (V) against time. Frequent regular apnoeas (A) and significant oscillations in carbon dioxide, oxygen and ventilation levels can be seen in FIGS. 11, 12a, 12b and 12c.

FIG. 12d shows the timing of supplying exogenous carbon dioxide (E.CO2). In this example, no exogenous carbon dioxide was supplied, and the oscillations in carbon dioxide, oxygen and ventilation levels continue over the whole time frame.

FIGS. 13a to 13d are equivalent to FIGS. 12a to 12d, though exogenous carbon dioxide is supplied part way through the test. Accordingly, FIG. 13d indicates points at which an exogenous carbon dioxide supply (E.CO2) was activated. A short delay between activation of the carbon dioxide supply and arrival of carbon dioxide in the lungs exists, but arrival of carbon dioxide in the lungs is timed to coincide with the troughs in end-tidal carbon dioxide which, as discussed with respect to FIG. 11, occurs after the dip in the carbon dioxide levels measured at the mouth. It can be seen that stabilisation of the periodic breathing occurs to a certain extent almost immediately, and that a significant reduction in the oscillations occurs within two cycles.

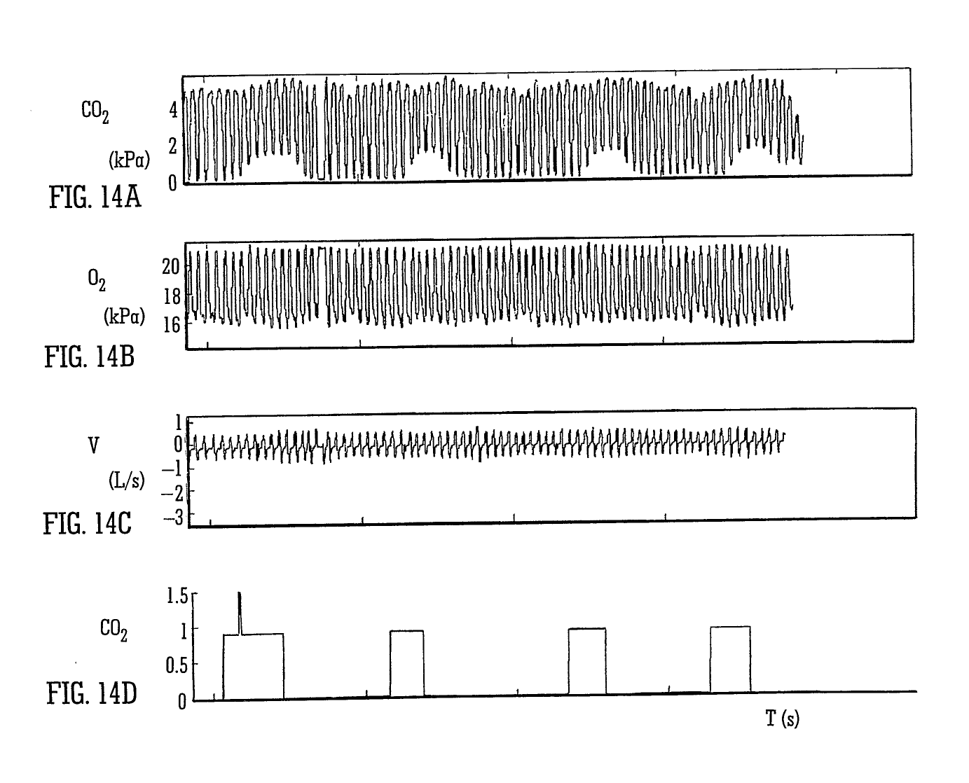

FIGS. 14a to 14d show the readings of the same patient after several consecutive cycles of treatment. Breathing patterns are substantially stable and minimal oscillations exist in the other respiratory parameters. Significantly, there are no apnoeas.

FIG. 15 shows the effect of dynamic changes to cardiac output using a pacemaker. FIG. 15a shows heart rate against time; FIG. 15b shows end-tidal carbon dioxide against time; and FIG. 15c shows end-tidal oxygen against time. The respiratory parameters of a patient can be seen to be stable while the heart rate was maintained at a constant level. Once alternations in heart rate were introduced via pacemaker re-programming, the patient demonstrated oscillations in end-tidal carbon dioxide and oxygen. Respiratory parameters stabilised once the heart rate was returned to a constant baseline value. Similar results are shown in FIG. 16a, though the variation caused in ventilation (V) is also shown. The readings over the five interventions recorded in FIG. 16a are superimposed and the mean and standard deviation is shown in FIG. 16b for each time point for each of end-tidal carbon dioxide, end-tidal oxygen and ventilation.

FIGS. 12a and 12b show that a patient's ventilation can be controlled by varying pacing parameters of a pacemaker.

1. A device for improving the stability of a ventilation pattern of a patient comprising
at least one sensor for sensing a parameter which reflects a level of lung gas in a patient and for producing an output signal indicative of said parameter, and
a processor adapted to receive and process the sensor output signal to assess the lung gas level, the processor being in communication with means for increasing the lung gas level of the patient, and being configured to produce a control signal for instructing said means in response to a decreasing level or a predicted decreasing level of lung gas, so as to retard a decrease in said lung gas level.

2. A device as claimed in claim 1, wherein the control signal instructs the lung gas increasing means such that the level of lung gas is increased at the point when the rate of decrease in the natural endogenous lung gas level is equal to or greater than a predetermined value.

3. A device as claimed in claim 1, wherein the processor is configured to identify a cyclic pattern of the lung gas level.

4. A device as claimed in claim 1, wherein the control signal is adapted to cause the output of the lung gas increasing means to follow a predetermined pattern.

5. A device as claimed in claim 4, wherein the pattern has a generally square, saw-tooth or sinusoidal profile.

6. A device as claimed in claim 1, wherein the control signal is adapted to cause the output of the lung gas increasing means to vary in response to real time variations detected in the lung gas level.

7. A device as claimed in claim 1, wherein the processor is configured to control said lung gas increasing means to have a maximum output so as to have a greatest effect on the lung gas level when the natural endogenous level of lung gas would, if untreated, be decreasing at its fastest rate.

8. A device as claimed in claim 1, wherein the control signal instructs the lung gas increasing means such that its output increases incrementally from one breathing cycle to the next breathing cycle.

9. A device as claimed in claim 8, wherein the control signal causes the output to remain constant from one breathing cycle to the next if an increase in output would destabilise breathing.

10. A device as claimed in claim 1, further comprising a memory unit to store the sensor output signal or a derivation thereof for access by the processor.

11. A device as claimed in claim 10, wherein the lung gas increasing means comprises a source of the lung gas in fluid communication with a delivery device configured to deliver the gas to a patient.

12. A device as claimed in claim 11, wherein the delivery device is a facemask or nasal cannula.

13. A device as claimed in claim 11, wherein the source is selected from the group consisting of: a pressurised canister or cylinder of the pure or dilute gas; an atmospheric pressure reservoir of the pure or dilute gas; and a reservoir of exhaled air collected from the patient.

14. A device as claimed in claim 11, further comprising a tube connected to the gas source and an electromechanical device associated with the tube, the control signal being adapted to operate the electromechanical device to adjust the pneumatic resistance of the tube.

15. A device as claimed in claim 11, further comprising a tube connected to the gas source and a valve associated with the gas source, the control signal being adapted to operate the valve to adjust the release of the gas from the source.

16. A device as claimed in claim 1, wherein the lung gas is carbon dioxide.

17. A device as claimed in claim 16, wherein the carbon dioxide increasing means comprises a pacemaker device, the operation of which is controlled by the control signal.

18. A device as claimed in claim 17, wherein the pacemaker is configured to increase a patient's heart rate in response to the control signal.

19. A device as claimed in claim 17, wherein the pacemaker is configured to pace a selected cardiac chamber in response to the control signal.

20. A device as claimed in claim 16, wherein the carbon dioxide increasing means comprises a hypoxic gas source.

21. A device as claimed in claim 16, wherein the carbon dioxide increasing means comprises an airflow control element adapted to adjust the degree of the patient's respiratory flow.

22. A device as claimed in claim 21, wherein the airflow control element is a physical restraint adapted to reduce the volume of breath taken in by the patient.

23. A device as claimed in claim 1, wherein the lung gas is oxygen.

24. A device as claimed in claim 23, wherein the oxygen increasing means comprises a pacemaker device, the control signal being adapted to operate the pacemaker to cause a reduction in a patient's cardiac output.

25. A device as claimed in claim 1, wherein the sensor and the processor are in communication via electrical wires or via wireless communication means.

26. A device as claimed in claim 1, wherein the processor and the lung gas increasing means are in communication via electrical wires or via wireless communication means.

27. A device as claimed in claim 1, wherein the sensor is one or more of selected from the group consisting of: a ventilatory sensor, a heart rate monitor, a blood velocity, heart rate or thoracic impedance monitor, a respiratory strain gauge, a blood carbon dioxide, oxygen, lactate or pH level monitor, an expired carbon dioxide or oxygen monitor, a thermistor or a peripheral oxygen saturation monitor, and a combination thereof.

28. A method of improving the stability of a ventilation pattern of a patient comprising:
detecting a parameter which reflects a level of lung gas in a patient; and
causing a retardation of a decrease in said level of lung gas in response to a decreasing level or predicted decreasing level of lung gas.

29. A method as claimed in claim 28, wherein the step of retarding a decrease in the level of the lung gas is commenced so as to cause a retardation of the decrease in the lung gas level at the point when the rate of decrease in the natural endogenous lung gas level is equal to or greater than a predetermined level.

30. A method as claimed in claim 28, further comprising identifying a cyclic pattern of the lung gas level.

31. A method as claimed in claim 28, wherein the step of retarding a decrease in the level of lung gas is carried out for a duration less than the period of the cyclic pattern.

32. A method as claimed in claim 28, wherein the retarding step involves a retarding force, the magnitude of which is determined according to a pre-set pattern.

33. A method as claimed in claim 32, wherein the pattern has a generally square, saw-tooth or sinusoidal profile with time.

34. A method as claimed in claim 28, wherein the retarding step involves a retarding force, the magnitude and duration of which is varied in response to real time variations detected in the lung gas level.

35. A method as claimed in claim 28, wherein the maximum retarding force is caused when the natural endogenous level of lung gas would, if untreated, decrease at its fastest rate.

36. A method as claimed in claim 32, wherein the magnitude of the retarding force is increased incrementally from one breathing cycle to the next breathing cycle.

37. A method as claimed in claim 36, wherein the retarding force remains constant from one breathing cycle to the next breathing cycle if an increase would destabilise breathing.

38. A method as claimed in claim 28, further comprising:
analysing the detected lung gas level over a period of time to determine the phase and amplitude of the lung gas cycle; and
comparing the phase and amplitude with reference phase and amplitude data to determine a suitable treatment regime.

39. A method as claimed in claim 38, wherein the comparison step includes interpolating the reference phase and amplitude data to the phase and amplitude of the detected signal.

40. A method as claimed in claim 38, wherein the reference phase, amplitude and treatment regime data are updated by the processor based on the patient's response to treatment.

41. A method as claimed in claim 40 wherein the processor can monitor the patient's response to treatment carried out to stabilise a breathing pattern.

42. A method as claimed in claim 40, wherein the processor can monitor the patient's response to doses of test treatment.

43. A method as claimed in claim 28, wherein the decrease in the level of lung gas in the lungs is retarded by delivering the lung gas to the patient from a source of the gas.

44. A method as claimed in claim 28, wherein the lung gas is carbon dioxide.

45. A method as claimed in claim 44, wherein the decrease in the level of carbon dioxide in the lungs is retarded by delivering a hypoxic gas mixture to the patient from a gas source.

46. A method as claimed in claim 44, wherein the decrease in the level of carbon dioxide in the lungs is retarded by varying the pacing parameters of a pacemaker to cause an increase in cardiac output.

47. A method as claimed in claim 28, wherein the lung gas is oxygen.

48. A method as claimed in claim 47, wherein the decrease in the level of oxygen in the lungs in retarded by varying the pacing parameters of a pacemaker to cause a reduction in cardiac output.

49. A method as claimed in claim 28—being carried out using the device of claim 1.
